(19) United States
(12) Patent Application Publication
(10) Pub. No.: US 2004/0143264 A1
(43) Pub. Date: Jul. 22, 2004

(54) Metal-backed UHMWPE rod sleeve system preserving spinal motion

(76) Inventor: Paul C. McAfee, Towson, MD (US)

Correspondence Address: Max Stul Oppenheimer, P.O. Box 50, Stevenson, MD, 21153, US

(21) Appl. No.: 10/645,202

(22) Filed: Aug. 21, 2003

Related U.S. Application Data
(60) Provisional application No. 60/405,775, filed on Aug. 23, 2002.

(51) Int. Cl. A61B 17/70

(52) U.S. Cl. 606/61

(57) ABSTRACT

A spinal rod sleeve system comprises attachment anchors having a plastic (preferably UHMWPE) sleeve which at least partially encircles a spinal rod, so as to allow a vertebra to slide cephalad or caudad along the spinal rod sleeve system. Preferably, said sleeve has a metal backed exterior surface, said rod has a hard outer surface suitable for gliding within said sleeve. The system helps preserve range of motion following spinal surgery.

This application is entitled to, and claims the benefit of, priority from U.S. Provisional Application Serial No. 60/405,775, filed Aug. 23, 2002

### FIELD AND BACKGROUND OF THE INVENTION

1. Field of the Invention

The present invention relates in general to prosthetic devices, and more specifically to a vertebral anchor spinal rod sleeve system.

2. Background Information

The invention described and claimed herein comprises a novel vertebral anchor spinal rod sleeve system that allows a vertebra to slide cephalad or caudad along a spinal rod system. The spinal rod sleeve system comprises attachment anchors having a plastic (preferably UHMWPE) sleeve which at least partially encircles a spinal rod, so as to allow a vertebra to slide cephalad or caudad along the spinal rod sleeve system. Preferably, said sleeve has a metal backed exterior surface, said rod has a hard outer surface suitable for gliding within said sleeve. The system helps preserve range of motion following spinal surgery.

Prior art systems for treating spinal problems which require a spinal rod sleeve system result in constraining the normal motion of the spine. As far back as 1983, Applicant recognized and began development of solutions to this problem. See, e.g., McAfee, Lubicky and Werner, “The Use of Segmental Spinal Instrumentation to Preserve Longitudinal Spinal Growth”, J. Bone & Jt. Surg (1983) (which describes the problem of treating long, progressive scoliotic curves in young patients while preserving longitudinal growth).

Prior attempts at solving this problem include DYNESES™, manufactured by Centerpulse Company; CISAD™, manufactured by Mekanika Company; and Edwards rod sleeves, manufactured by Zimmer Company.

Dynesys™ is a posterior motion fixation system with polycarbonate-polyurethane and a central elastic cord. This device has several disadvantages. There is no sliding motion within the hook, screw or anchor to the patient's spine. The only motion occurs between the vertebral levels. The rod or longitudinal member does not change its orientation cephalad or caudad to the individual verebra.

The Mekanika device utilizes a carbon fiber flexible rod which does not slide at its point of fixation to the spine.

The Zimmer Edwards rod sleeve is made of Ultra High Molecular Weight Polyethylene (“UHMWPE”), but is not metal backed.

Furthermore, it does not allow motion of the rod. It was approved (and intended to be used) solely as a fusion device for fracture fixation. The rod sleeves were never used as a fixation device at the level of the spinal vertebra; instead, they were used for fractures, to provide a third or fourth point of pressure at the posterior elements of the fractured vertebral level. They do not preserve or allow spinal motion or flexion, extension or bending.

Rivard (U.S. Pat. No. 6,554,831) describes a system for preserving a degree of spinal motion. However, the system is all metal, and will result in debris and will bind, thereby restricting motion and, in general, will result in many of the problems described in the above-cited 1983 article and generally recognized in the art.

See, for example:

1. Archibeck MJ, Jacobs JJ, Roebuck KA, et al. The basic science of periprosthetic osteolysis. J Bone Joint Surg [AM] 2000;(82-A): 1478-1489.

2. Doorn PF; Campbell Pa.; Amstutz HC. Metal versus polyethylene wear particles in total hip replacements. A review. Clin Orthop 1996;(329 Suppl):S206-216.

3. Doorn PF; Mirra JM; Campbell Pa.; Amstutz HC: Tissue reaction to metal on metal total hip prostheses. Clin Orthop 1996;(329 Suppl):S187-205.

4. Gaine WJ, Andrew SM, Chadwick P et al: Late Operative Site Pain with ISOLA Posterior Instrumentation Requiring Implant Removal. Infection or metal reaction? Spine 2001 26:583-587.

5. Dubousset J, Shufflebarger H, Wenger D. Late “infection” with C-D instrumentation. (Abstract) Orthopaedic Transactions 1994;18:121.

Furthermore, as shown in FIG. 3 of the Rivard patent (and described at column 4, lines 49-64), a roller element is required in order to facilitate motion and this requires a gap for rotation. This adds shucking and increases the chances of loosening. Gaps between components should only occur where the sliding motion is supposed to take place and that is at the rod vs rod-sleeve interface.

Moreover, the Rivard device provides an offset between the longitudinal axis of the screw and the longitudinal axis of the rod; by providing a device where the application of the longitudinal rod tracks over the vertebral pedicle, the invention described herein reduces the torque and binding friction between components, thereby providing greater range of motion.

### SUMMARY OF THE INVENTION

The foregoing problems are overcome, and other advantages are provided by a spinal rod sleeve system comprising attachment anchors having a sleeve of Ultra High Molecular Weight Polyethylene (“UHMWPE”) which fully, or at least partially, encircles a spinal rod, so as to allow a vertebra to slide cephalad or caudad along the spinal rod system.

Among the objects of the invention are to provide a spinal rod sleeve system comprising attachment anchors having a sleeve which at least partially encircles a spinal rod, so as to allow a vertebra attached thereto to slide cephalad or caudad along the spinal rod system.

Another object of the invention is to provide a spinal rod sleeve system comprising a longitudinal spinal rod core, having as a second layer a concentric circle of UHMWPE, plastic or other suitable material, and an outer layer of a suitable metal (for example, stainless steel, cobalt chrome or titanium alloy), suitable for clamping or anchoring the system to a patient's vertebra.

The various features of novelty which characterize the invention are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

The foregoing and still other objects of this invention will become apparent, along with various advantages and features of novelty residing in the present embodiments, from study of the following drawings, prepared at the inventor's direction, in which:

FIGS. 1-3 illustrate the rod-sleeve system in place in a patient's spine, attached by optional methods: FIG. 1 illustrates attachment by sublaminar wires; FIG. 2 illustrates attachment by pedicle screws placed so as to achieve nerve root decompression; FIG. 3 illustrates attachment by pedicle screws placed so as to eliminate torque.

FIGS. 4 and 5 illustrate fully-constrained and unconstrained options.

FIG. 6 illustrates the components and construction of a one-piece non-slotted rod connector.

FIG. 7 illustrates the components and construction of a pedicle screw in accordance with the invention.

FIG. 8 illustrates the details of a split connector.

FIG. 9 illustrates construction details of a metal sleeve connector suitable for press-fitting a UHMWPE sleeve.

FIG. 10 shows top and end views of a UHMWPE spool, suitable for slip fitting over a rod.

FIG. 11 shows the embodiment of the invention as a bumper.

### DESCRIPTION OF THE PREFERRED EMBODIMENT

Referring to the drawings, the invention is a novel spinal rod sleeve system comprising attachment anchors having a UHMWPE sleeve which at least partially encircles a spinal rod, so as to allow a vertebra to slide cephalad or caudad along a spinal rod sleeve system.

A spinal rod sleeve system is provided with attachments (anchors) to a patient's spine. These anchors may be attached to spinal lamina, spinous processes, pedicles or posterior elements of the spine, and commonly include (but are not limited to) hooks, screws or wires.

The rod sleeve has an outer surface, preferably of metal, which serves as a containment casing, and an inner surface, preferably of plastic, said inner surface of the sleeve being the outer bearing surface of the system. The sleeve encircles a longitudinal rod having an external surface which serves as the inner bearing surface of the system. In combination, the rod and sleeve allow sliding or gliding movement between the outer and inner bearing surfaces.

In a preferred embodiment, the application of the longitudinal rod tracks over the vertebral pedicle, so as to minimize torque and binding friction between components, thereby providing greater range of motion.

The anchors comprise a sleeve or bushing, preferably made of UHMWPE, plastic or other suitable material, which encircles (either partially or completely) a spinal rod. Preferably, said sleeve is metal backed. Suitable metals include stainless steel, cobalt chrome or titanium alloy.

A longitudinal spinal rod core comprises an inner layer of a nonmetallic material, preferably a plastic, and most preferably UHMWPE, polyethylene or high density polyethylene, surrounded by a second concentric layer of UHMWPE, plastic or other suitable material, and an outer layer of a suitable metal (for example, stainless steel, cobalt chrome or titanium alloy).

In order to allow the spinal rod to slide or telescope, the encircling sleeve allows sliding along its inner diameter; the encasing outer diameter of the sleeve is encircled, clamped or otherwise fixed to a metal attachment.

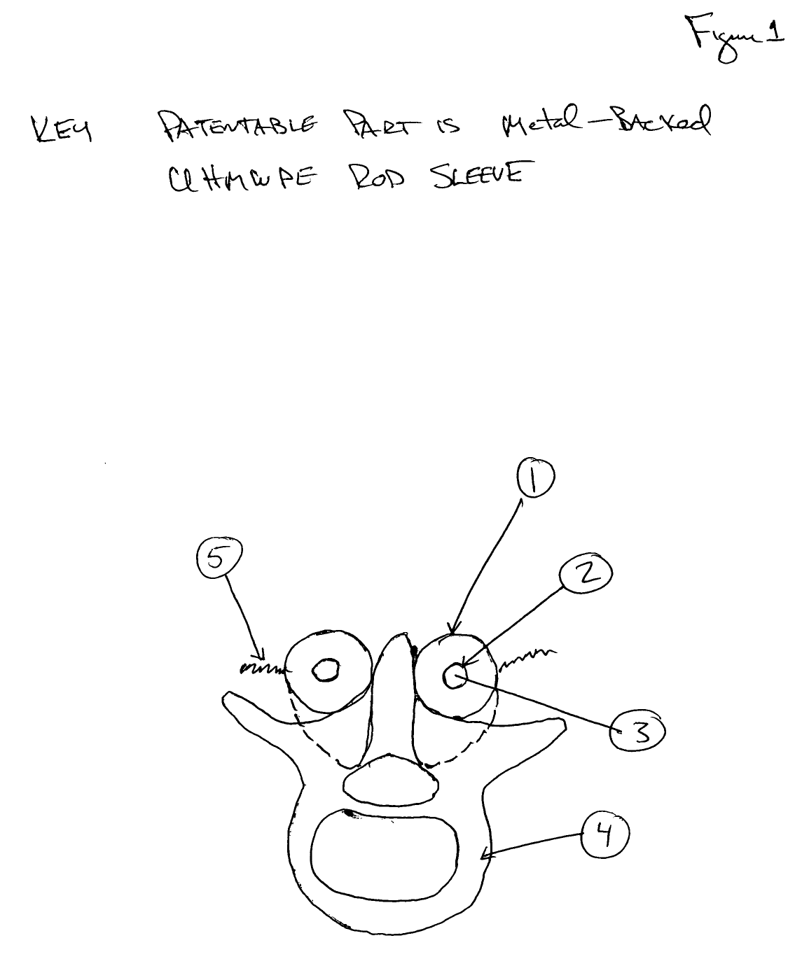

Referring to FIG. 1, the elements and attachment of the spinal rod sleeve system may be seen. The sleeve or bushing is cylindrical in shape (and may be continuous or c-shaped) and (viewed in cross-section) has an external surface (1) and an interior or bearing surface (2) within which a spinal rod (3) fits.

The system is attached to a patient's spine (4) using suitable anchoring means known to those skilled in the art—in FIG. 1 (by way of illustration), sub laminar wires (5), but other bone anchors (7) could include screws, pedicle screws, wires, sublaminar wires or hooks.

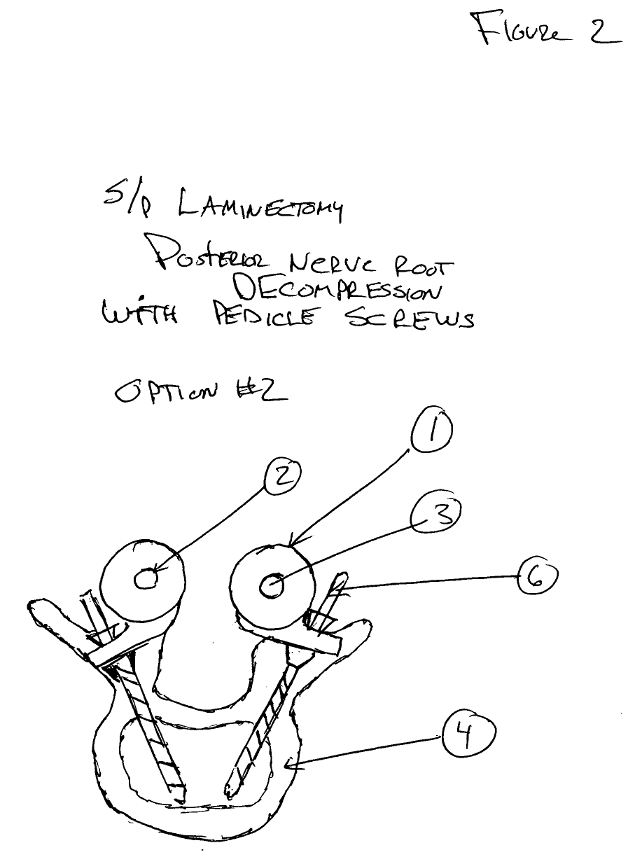

FIG. 2 illustrates use of the spinal rod system for posterior nerve root decompression using pedicle screws (6).

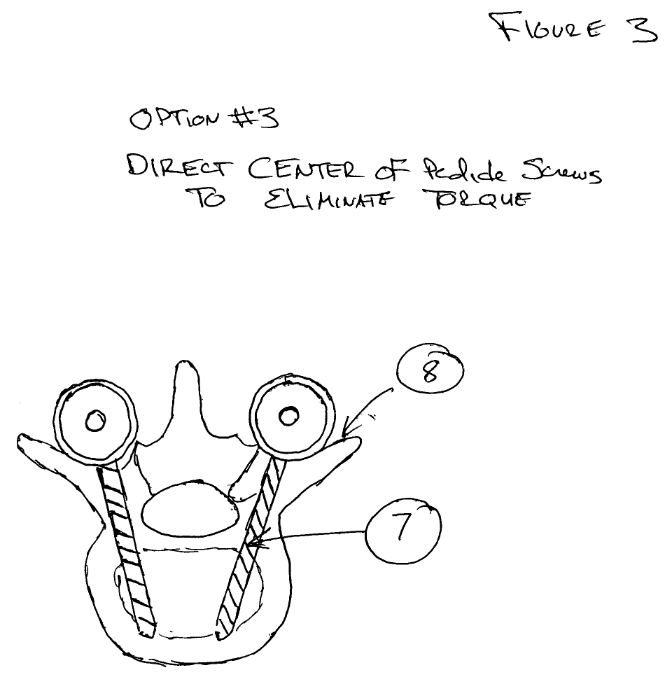

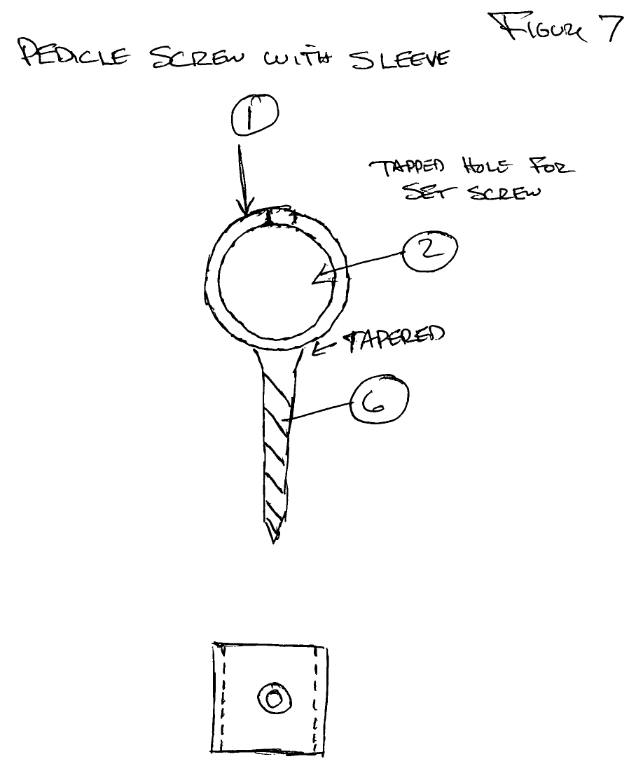

As shown in FIG. 3 (and an alternative in FIG. 7), the longitudinal rod is preferably attached so that it tracks over the vertebral pedicle (8), allowing the axis of the screw and rod to be intersecting and minimizing or eliminating any offset between the longitudinal axis of the screw and the longitudinal axis of the rod, thus reducing torque and thereby reducing binding friction between the gliding surfaces and improving motion.

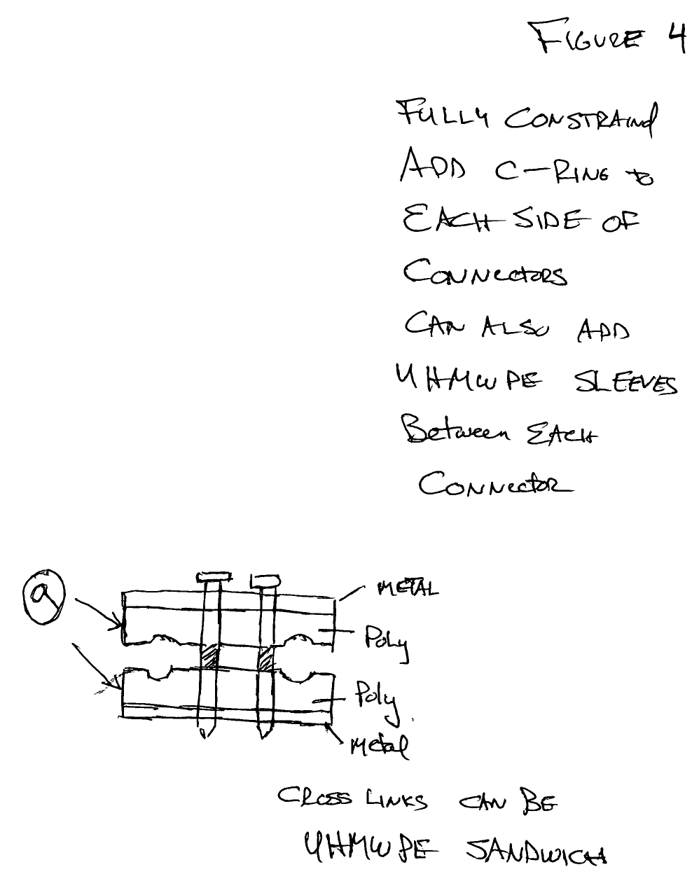

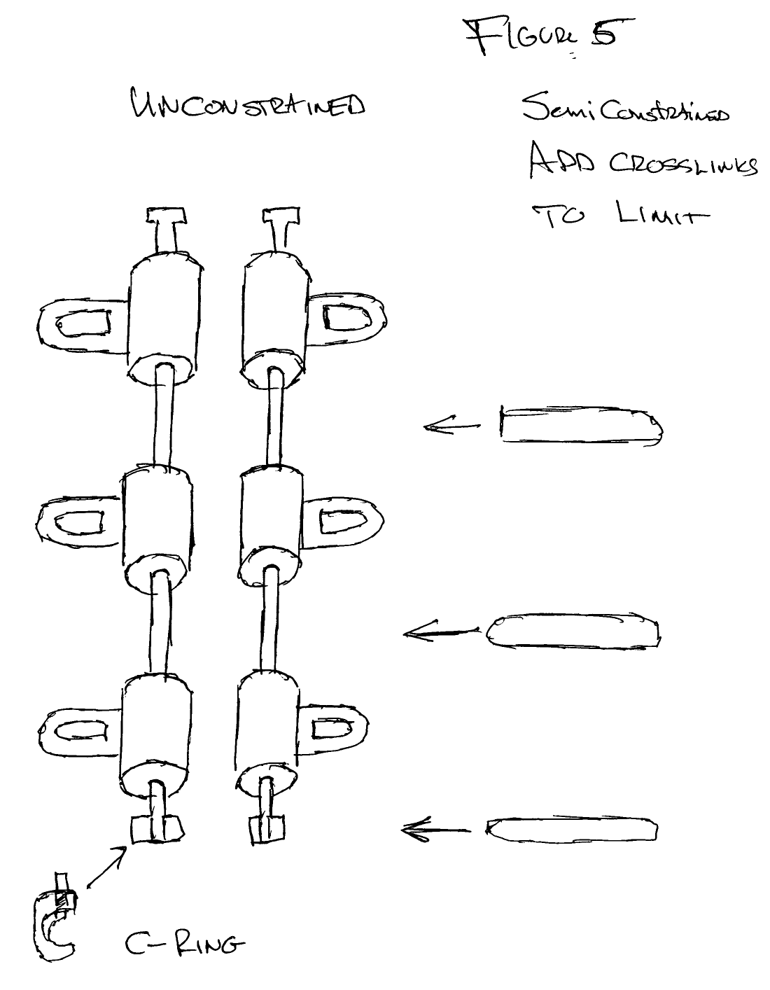

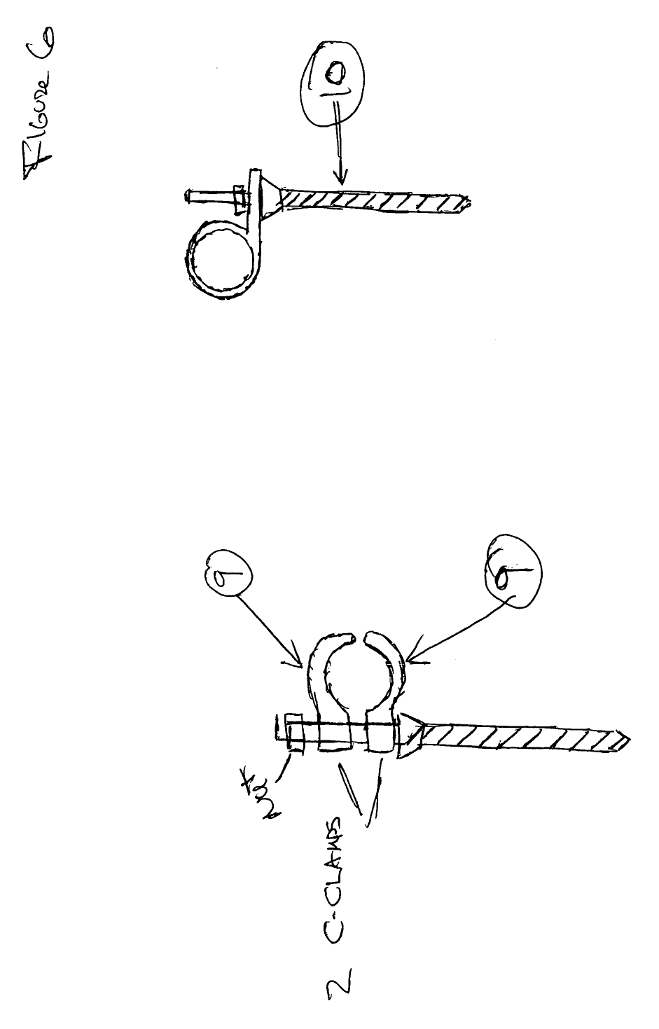

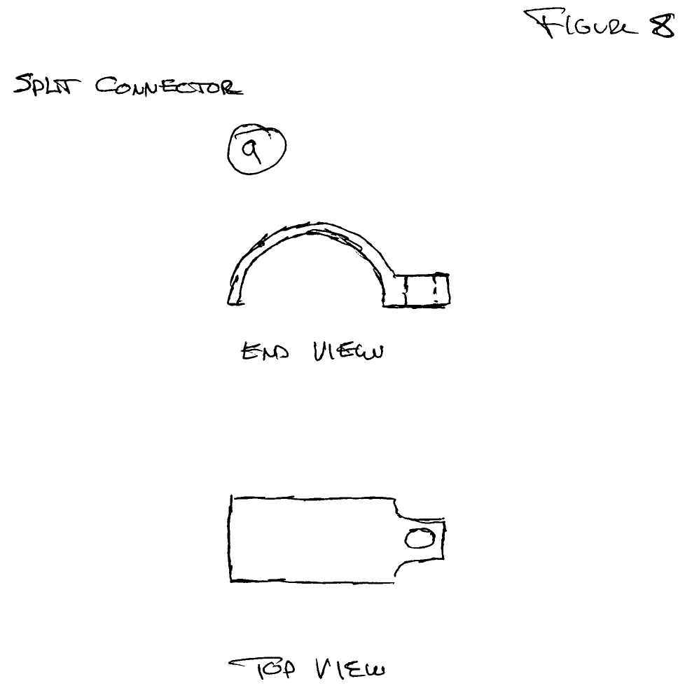

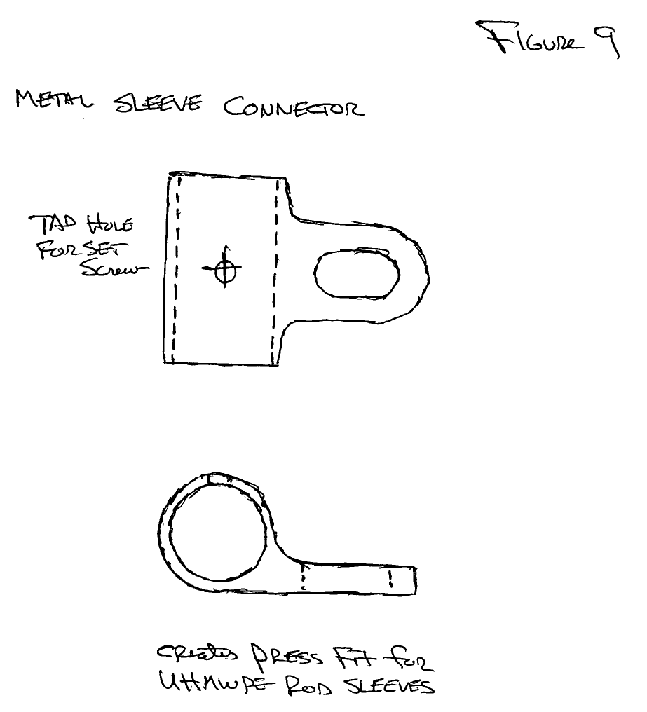

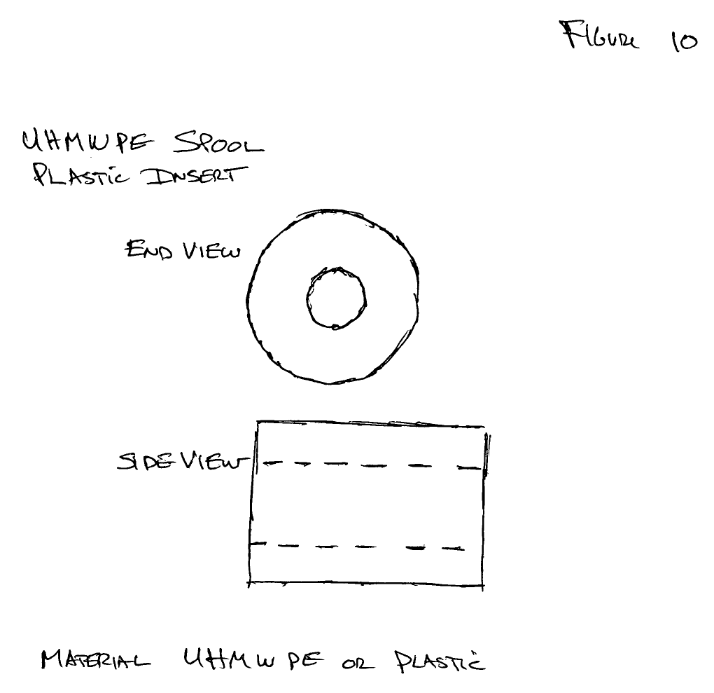

Preferred construction details of components of the spinal rod sleeve system are shown in FIGS. 4-10. Note that (as shown in FIG. 6) the rod connector may be solid, slotted (10), or composed of two opposing c-clamps (9). Ideally, the longitudinal rod is made of a hard material such as metal, and the surfaces coming into contact with the rod have a plastic or similar gliding surface. The gliding surface, such as the rod sleeve, has a layer of softer material such as plastic or UHMWPE in contact with the rod. The next outer layer providing a casing around or surrounding the plastic is also a harder material which provides attachment to the bony vertebra.

More generally, the spinal rod sleeve system can be used in treating a spinal disorder whose treatment would benefit from allowing a vertebra to slide cephalad or caudad along a spinal rod sleeve system, or otherwise preserving spinal motion, by anchoring such a system to a patient's spinal lamina, spinous processes, pedicles or posterior elements of the spine. The internal bearing layer around the rod allows gliding motion between the rod and the inner surface of the sleeve; using low-friction materials facilitates motion approaching that of a normal spine.

Anchoring the system to bone using a rotating (i.e., “polyaxial”) or fixed (i.e., “monoaxial”) attachment permits the adjacent vertebrae to get closer together or farther apart.

As compared to metal to metal surfaces, the disclosed invention provides a lower coefficient of friction. The difference is more pronounced if the surfaces are non concentric—i.e., if the outer metal sleeve doesn't exactly conform to the longitudinal rod because the inner rod needs to be bent to conform to the patient's normal lumbar lordosis and normal thoracic kyphosis. Since by definition the two bearing surfaces in the spine are not going to be concentric they will not be amenable to a metal-on-metal bearing surface or inner metal surface on the rod sleeve.

The bone anchor may be a differentially locking polyaxial screw which attaches to the longitudinal rod; this allows differential polyaxial movement or could be locked differentially to different motions. For example, it could allow flexion/extension but prevent anterior vertebral translation, or it could maintain sagittal alignment of fixation yet prevent spinal flexion, extension or bending, or it could allow rotation but not allow rocking or sliding down the longitudinal axis of the rod.

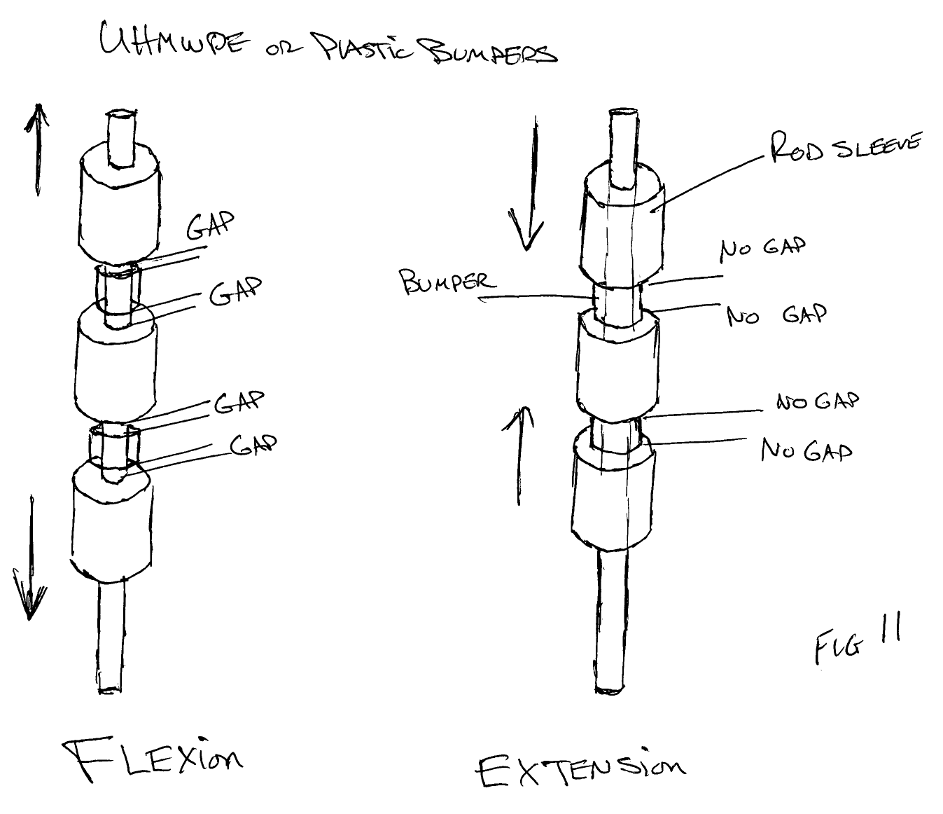

As shown in FIG. 11, the UHMWPE sleeves or blockers can also function as blockers or bumpers to dampen excessive spinal extension movement.

Alternative embodiments utilizing the underlying invention include a metal backed rod sleeve (preserving spinal motion), sublaminar wires attaching the metal backed UHMWPE rod sleeve, pedicle screws directly incorporating UHMWPE rod sleeves, slotted or offset rod connectors attaching pedicle screws to metal backed UHMWPE rod sleeves, hooks attaching to vertebra and incorporating a metal backed UHMWPE rod sleeve, and transverse rod connector fabricated as a sandwich having an outer layer of metal or other suitable material and an inner layer of plastic (preferably UHMWPE) or other material suitable for bearing on a spinal rod so as to enable cephalad or caudad sliding motion, as shown in FIGS. 4-7.

More generally, the invention may be used in any procedure where allowing a vertebra to slide cephalad or caudad along a spinal rod sleeve system, or otherwise preserving spinal motion, is desirable.

Furthermore, the invention may be adapted for use in other applications requiring a layered connection with a harder outside casing with a softer inner core which articulates with the longitudinal (harder material) rod, for example low friction arthroplasty as described by Sir John Charnley (see, e.g., Charnley, John-Total hip replacement by low friction arthroplasty.

Clinical Orthopaedics and Related research 72:7, 1970; Charnley, J, and Cupic, Z. The nine and ten year results of the low friction arthroplasty of the hip. Clinical Orthopaedics and Related Research, 95:9, 1973; Charnley, J, and Feagin, J.: Low friction arthroplasty in congential subluxation of the hip. Clinical Orthopedics and Related Research 91: 98, 1973; and Charnley, J and Halley, DK: Rate of Wear in Total Hip Replacement, Clinical Orthopedics ad Related Research 112:170, 1975) whereby motion is facilitated by use of a hard material articulating with a softer material.

While a specific embodiment of the invention and several variations have been shown and described in detail to illustrate the application of the principles of the invention, it will be understood that the invention may be embodied otherwise without departing from such principles and that various modifications, alternate constructions, and equivalents will occur to those skilled in the art given the benefit of this disclosure. Thus, the invention is not limited to the specific embodiment described herein, but is defined by the appended claims.

I claim:

1. A spinal rod sleeve system comprising a longitudinal spinal rod disposed within a concentric sleeve, said sleeve having an internal bearing layer and an external layer.

2. A system as in claim 1 wherein said internal bearing layer is a low-friction material.

3. A system as in claim 1 wherein said internal bearing layer is a plastic.

4. A system as in claim 1 wherein said internal bearing layer is UHMWPE.

5. A system as in claim 1 wherein said external layer is a metal.

6. A system as in claim 1 wherein said external layer is a material selected from the group consisting of stainless steel, stainless steel alloys, cobalt chrome, cobalt chrome alloys, titanium and titanium alloys.

7. A method for treating a spinal disorder whose treatment would benefit from allowing a vertebra to slide cephalad or caudad along a spinal rod sleeve system, or otherwise preserving spinal motion, comprising providing a spinal rod sleeve system as in claim 1 and anchoring said spinal rod sleeve system to a patient's spinal lamina, spinous processes, pedicles or posterior elements of the spine.

8. A method for treating long, progressive scoliotic curves in young patients while preserving longitudinal growth, comprising the steps of providing a spinal rod sleeve system as in claim 1 and anchoring said spinal rod sleeve system to a patient's spinal lamina, spinous processes, pedicles or posterior elements of the spine.

9. A system as in claim 1, wherein said spinal rod is free to glide within said sleeve.

10. A system as in claim 1, further comprising an anchor to a bone, to which said sleeve is attached.

11. A system as in claim 10, further comprising a bumper or sleeve on the connecting rod.

12. A system as in claim 11 wherein said bumper or sleeve is placed so as to restrict spinal extension to a desired range.

13. A system as in claim 12 wherein said desired range is chosen so as to maintain a desired distance between rod sleeves.

14. A system as in claim 11 wherein said anchor is selected from among the group consisting of screws, pedicle screws, wires, sublaminar wires and hooks.

15. A method as in claim 7, further comprising establishing a polyaxial (or rotating) or monoaxial (or fixed) attachment to the rod which selects out unwanted motion directions thereby allowing variation of the distance between vertebrae.

16. A spinal rod for a spinal rod sleeve system comprising a longitudinal spinal rod having an inner core and an outer bearing surface, said inner core being comparatively hard in relation to said outer bearing surface, and said outer bearing surface being composed of UHMWPE.

17. A spinal rod sleeve for a spinal rod sleeve system comprising a sleeve having an outer casing surface and an inner bearing surface, said outer casing surface being comparatively hard in relation to said inner bearing surface, and said inner bearing surface being composed of UHMWPE.

18. A system for low friction arthroplasty, comprising a core disposed within a concentric sleeve which is harder than said core, so as to facilitate motion by articulation of said core with said concentric sleeve.
